(19) United States
(12) Patent Application Publication
(10) Pub. No.: US 2025/0249150 A1
(43) Pub. Date: Aug. 7, 2025

(54) HYDROGELS FOR FILLING SOFT TISSUES

(71) Applicant: TEOXANE SA, GENEVE (CH)

(72) Inventors: Jimmy FAIVRE, VALSERHONE (FR); François BOURDON, GAILLARD (FR)

(21) Appl. No.: 18/857,051

(22) PCT Filed: Apr. 14, 2023
(86) PCT No.: PCT/EP2023/059829

§ 371 Date: Oct. 15, 2024

(30) Foreign Application Priority Data
Apr. 15, 2022 (FR) FR2203536
Apr. 15, 2022 (FR) FR2203541
Dec. 23, 2022 (FR) FR2214438

(51) Int. Cl. A61L 27/52; A61L 27/20; A61L 27/54

(52) U.S. Cl. CPC A61L 27/52; A61L 27/20; A61L 27/54

(57) ABSTRACT

The present disclosure relates to hydrogels based on crosslinked polysaccharides having ability to create moderate volumes which are maintained over time. The hydrogels have a modification ratio less than or equal to 1% and a projection index PIdx ranging from 50 to less than 80%.

### TECHNICAL FIELD

The present invention relates to filler gels, in particular based on crosslinked polysaccharides, used in the aesthetic and/or medical field for filling wrinkles and/or giving more relief to certain areas of the face.

### PRIOR ART

The filler gels are, for example, polysaccharide-based hydrogels, in particular hyaluronic acid (HA), and are injected under the skin using a syringe.

Hydrogels suitable for filling soft tissues are typically crosslinked hydrogels, i.e., the polysaccharide is crosslinked by means of one or more crosslinking agents. This crosslinking makes it possible to obtain a hydrogel with the mechanical properties desirable for filling soft tissues.

Manufacturers propose filler hydrogels having various rheological properties depending on the injection site of the hydrogel. For example, in the case of injection into the outer layers of the skin, the hydrogels should lightly fill the fines wrinkles and fine lines and be capable of easily following facial movements. Whereas hydrogels intended for deep implantation, in particular intended for filling more severe folds and wrinkles and/or for creating volume, must advantageously have an intrinsic ability to sustainably maintain their thickness in the layers of the skin, including under stress from facial movements. For intermediate applications, for example injections into the mid-dermal layers of the skin, gels having intermediate properties are chosen, in other words properties between the properties of gels intended for deep applications and the properties of gels intended for superficial applications.

The most usually exploited rheological measurements of hydrogels are measurements of the G′ and G″ moduli, the phase-shift angle δ being related to these measurements (tan δ=G″/G′). These measurements are commonly carried out under a low oscillatory stress with a low amplitude, in their linear viscoelastic region. These measurements do not necessarily reflect the mechanical stress and deformation that a filler hydrogel undergoes in vivo. These measurements do not make it possible to predict the behaviour under compression of the hydrogel. However, it is important to be able to predict the behaviour of the hydrogels in situ in order to guarantee that the sought-after effect has been obtained, particularly when the filler gel is intended for the creation of moderate/subtle volume.

Commercial so-called “volumiser” hydrogels intended for the creation of volume are crosslinked hydrogels, prepared with non-negligible quantities of crosslinking agent.

The crosslinked hydrogels may have biocompatibility problems. In order to optimise this biocompatibility, one solution consists of reducing the degree of modification of the polysaccharide necessary for obtaining the gel in order to be the closest to the natural polysaccharide or to opt for alternative crosslinking methods, such as crosslinking by sol-gel reaction. However, with such solutions, the hydrogels prepared cannot have mechanical properties suitable for use as volumisers, in particular these hydrogels are likely to be less crosslinked, may have a reduced G′ modulus and a low ability to maintain their thickness in the tissues.

There is therefore a need to provide hydrogels, in particular volumiser hydrogels, having a minimal degree of modification of the polysaccharide, and nevertheless having mechanical properties suitable for the creation of volume and sustainably maintaining their thickness in the layers of the skin, including under stress from facial movements.

The present invention thus aims to provide crosslinked polysaccharide-based hydrogels which have a volumising capability, in particular suitable for the creation of moderate/subtle volume, while being highly biocompatible, in other words also natural and also modified as little as possible.

### SUMMARY OF THE INVENTION

The invention relates to a crosslinked polysaccharide-based hydrogel having a modification ratio less than or equal to 1% and having a projection index PIdx ranging from 50% to less than 80%, the projection index being determined according to the method described in detail below.

The invention also relates to a crosslinked polysaccharide-based hydrogel having a modification ratio less than or equal to 1% and having a projection index PIdx ranging from 50% to less than 80%, prepared by a method comprising the following steps:
a) providing at least one polysaccharide or a salt thereof;
b) providing at least one crosslinking agent or a salt thereof;
c) preparing a crosslinking reaction medium comprising the one or more polysaccharides, the one or more crosslinking agents and a solvent, the total quantity of crosslinking agent ranging from 0.001 to 0.01 mole per 1 mole of polysaccharide repetition unit, the duration of the preparation step of the reaction medium preferably not exceeding 5 hours; and
d) placing the reaction medium obtained at the end of step c), at a pressure P less than or equal to atmospheric pressure and at a temperature T greater than the temperature of the eutectic point of the reaction medium as measured at pressure P and less than the temperature of the freezing point of the reaction medium as measured at pressure P, preferably for a duration ranging from 1 week to 17 weeks.

The invention also relates to a crosslinked polysaccharide-based hydrogel having a modification ratio less than or equal to 1% and having a projection index PIdx ranging from 50% to less than 80%, prepared by a method comprising the following steps:
a) providing at least one polysaccharide or a salt thereof;
b) providing at least one functionalisation agent or a salt thereof, as described below;
c) preparing a crosslinking reaction medium comprising the one or more polysaccharides, the one or more functionalisation agents and a solvent, the total quantity of functionalisation agent ranging from 0.001 to 0.01 mole per 1 mole of polysaccharide repetition unit, the duration of the preparation step of the reaction medium preferably not exceeding 5 hours; and
d) placing the reaction medium obtained at the end of step c), at a pressure P less than or equal to atmospheric pressure and at a temperature T greater than the temperature of the eutectic point of the reaction medium as measured at pressure P and less than the temperature of the freezing point of the reaction medium as measured at pressure P, preferably for a duration ranging from 1 week to 17 weeks.

The invention also relates to the use of the hydrogels of the invention for preventing and/or treating alterations in viscoelastic or biomechanical properties of the skin; to fill wrinkles, fine lines and scars; to reduce nasolabial folds and bitterness folds; to increase the volume of cheekbones, of the chin or lips or to reduce the appearance of wrinkles and fine lines.

Other aspects of the invention are as described in the claims and below.

### Definitions

The term “gel” designates a polymer network which is dilated over its entire volume by a fluid. This means that a gel is formed of two media, one “solid” and the other “liquid”, dispersed in one another. The medium referred to as “solid” consists of long-molecule polymers connected to one another by weak bonds (for example hydrogen bonds) or covalent bonds (crosslinking). The liquid medium consists of a solvent. A gel generally corresponds to a product which has a phase angle δ less than or equal to 45° at 1 Hz for a deformation of 0.1% or a pressure of 1 Pa, advantageously a phase angle δ ranging from 2° to 45° or from 20° to 45°.

The term “hydrogel” designates a gel as defined above, wherein the solvent constituting the liquid medium is mostly water (for example at least 90%, in particular at least 95%, especially at least 97%, especially at least 98% by weight of the liquid medium). Preferably, the liquid medium comprises, in particular consists of, a buffer solution, advantageously enabling a pH of the liquid medium between 6.8 and 7.8, in particular a phosphate-buffered saline.

The term “injectable gel” designates a gel that can flow and be injected manually by means of a syringe equipped with a needle of diameter ranging from 0.1 to 0.5 mm, for example a 32 G, 30 G, 27 G, 26 G, 25 G hypodermic needle. Preferably, an “injectable gel” is a gel having a mean extrusion force less than or equal to 25 N, preferably ranging from 5 to 25 N, more preferably ranging from 8 to 15 N, during a measurement with a dynamometer, at a fixed speed of approximately 12.5 mm/min, in syringes of external diameter greater than or equal to 6.3 mm, with a needle of external diameter less than or equal to 0.4 mm (27 G) and length ½″, at ambient temperature.

A “superficial application” designates the superficial administration, for example by mesotherapy, of a composition into the skin, or on the skin, for treatment of the outer layers of the skin, the epidermis and the most superficial parts of the dermis, in order to reduce superficial wrinkles and/or to improve the quality of the skin (such as its radiance, density or structure) and/or to rejuvenate the skin.

A “mid-dermal application” designates the administration of a composition in the mid-dermal part of the skin in order to treat the mid-dermal layers of the skin, as well as to reduce the mid-dermal wrinkles.

A “deep application” designates the administration of a hydrogel in the deepest layers of the skin, the hypodermis and the deepest part of the dermis, and/or under the skin (above the periosteum) for “volumising” the soft tissues, such as for filling the deepest wrinkles and/or partially atrophied areas of the facial and/or body contour. So-called “volumiser” hydrogels can typically be administered for deep application.

A “crosslinked polysaccharide” designates a polysaccharide modified during a crosslinking reaction. The crosslinking may be carried out by means of a crosslinking agent or results from a sol-gel reaction. When the crosslinking reaction is a sol-gel reaction, the polysaccharide is modified with a functionalisation agent.

Conversely, a “non-crosslinked polysaccharide” designates a polysaccharide that is not modified with a crosslinking agent and/or functionalisation agent and which therefore has not undergone a crosslinking reaction.

The term “crosslinking agent” designates any compound capable of introducing crosslinking between different polysaccharide chains.

The term “functionalisation agent” designates any compound capable of reacting with a functional group of the polysaccharide and of reacting via a sol-gel reaction.

The “molar crosslinking ratio” (TR), expressed in %, designates the molar ratio of the quantity of crosslinking agent to the quantity of polysaccharide repetition unit introduced into the crosslinking reaction medium, expressed for 100 moles of polysaccharide repetition units in the crosslinking medium.

The “molar functionalisation ratio”, expressed in %, designates the molar ratio of the quantity of functionalisation agent to the quantity of polysaccharide repetition unit introduced into the crosslinking reaction medium, expressed for 100 moles of polysaccharide repetition units in the functionalisation medium.

The expression “repetition unit” of a polysaccharide designates a structural unit consisting of one or more (generally 1 or 2) monosaccharides, the repetition of which produces the complete polysaccharide chain. 15 The “degree of modification” (MOD) of a polysaccharide, such as hyaluronic acid, corresponds to the molar quantity of crosslinking agent bonded to the polysaccharide and/or the functionalisation agent bonded to the polysaccharide, by one or more of its ends, expressed for 100 moles of polysaccharide repetition units. It can be determined by the methods known to a person skilled in the art, such as nuclear magnetic resonance spectroscopy (NMR) (see experimental section).

The term “polysaccharide” designates a polymer composed of monosaccharides (preferably D-enantiomers) joined together by glycosidic bonds.

The term “ambient temperature” shall mean a temperature ranging from 20 to 25° C., more particularly 25° C.

### DETAILED DESCRIPTION OF THE INVENTION

The proposed crosslinked polysaccharide-based hydrogels are characterised by a modification ratio less than or equal to 1% and a projection index PIdx ranging from 50 to less than 80%, preferably ranging from 60 to less than 80%, the projection index being determined as described in detail below. The projection index reflects the rheological behaviour of the hydrogels under a force over time. In other words, such hydrogels have the ability to maintain a thickness greater than or equal to 50% and less than 80% of their initial thickness over time.

The hydrogels for which the projection index PIdx varies from 50 to less than 80% have an ability to create moderate/subtle volumes which are maintained over time.

The projection index therefore makes it possible to discriminate between hydrogels, in order to retain that or those having the most interesting properties for the desired result, in particular, when seeking to obtain so-called “volumiser” hydrogels intended for volume creation, in particular “volumiser” hydrogels used for creating moderate/subtle volume.

The hydrogels of the present invention, by virtue of this projection index ranging from 50% to less than 80%, prove to be good versatile filler gels, in other words useful for the correction of moderate defects.

### Determining the Projection Index

The projection index is determined by a method comprising the following steps:
1) subjecting a bolus of one gram of hydrogel to a fixed constant compression force F of 2 newtons, said hydrogel being disposed beforehand between two circular pressure plates of a rheometer and having an initial thickness do of 700 μm given by the distance between the two plates, the two plates being parallel and having a diameter of 40 mm,
2) acquiring, at 25° C., the development of the variation of the thickness of the hydrogel compressed in this way for 1 hour;
3) parameterising a generalised Maxwell model approximating the development observed from the acquisition carried out in step 2), said model expressing the thickness of the hydrogel as a function of time d_{gel}(t) and responding to the following equation 1:

$${d}_{gel} ( t ) = {d}_{∞} + \sum {A}_{i} \cdot {e}^{( - t / {τ}_{i} )} \qquad ( equation 1 )$$

where do_{∞} is the limit thickness obtained at equilibrium, Aᵢ is a constant and τi a relaxation parameter, the number of members i of the equation being equal to 2;
4) determining, from the model, a limit thickness d_{∞} towards which the hydrogel tends to develop over time; and
5) generating the projection index PIdx from the following equation 2:

$$PIdx ( \% ) = \frac{{d}_{∞}}{{d}_{0}} \cdot 100. \qquad ( equation 2 )$$

The limit thickness obtained at equilibrium (d_{∞}) designates the thickness obtained at infinite time for the applied compression force F.

The applied compression force F (2 N) is preferably in the region for linear viscoelastic deformation of the hydrogel, in other words the applied compression force F is advantageously manifest as a pressure in the linear viscoelastic region of the hydrogel. Such a force value is suitable for so-called volumiser filler gels. The linear viscoelastic region (LVER) corresponds to the range of hydrogel deformations ranging from an initial value of elastic modulus E′ to the value of the elastic modulus E′ reduced by 10% from its initial value.

The method for determining the projection index can thus initially comprise a step of determining the linear viscoelastic region of the hydrogel. This step can ensure that the applied compression force F is indeed in the region of linear viscoelastic deformation of the gel. Advantageously, this step consists of an oscillatory stress scanning measurement in compression mode at a given oscillation frequency, in order to determine the linear viscoelastic region and to set limits on the applied normal force. This measurement is applied over a range of stress determined under conditions identical to the measurement conditions for the projection index.

Preferably, the stress range covers from 10 Pa to 10,000 Pa at 1 Hz and 25° C., the plate geometry is parallel plates (diameter 40 mm anodised aluminium, TA Instruments®), and the distance between the base plate and the pressure plate is equal to 700 μm.

When the applied compression force F is in the linear viscoelastic region LVER of the hydrogels, the projection index PIdx is a direct measurement of their ability to maintain their initial thickness under a given compression stress. The projection index thus reflects the behaviour of the hydrogel within tissues.

When the applied compression force F is beyond their linear viscoelastic deformation region LVER, the projection index PIdx gives an indication regarding their ability to keep their thickness over a certain period; in theory, in an unconfined geometry, these hydrogels would continue to creep, but in reality the surrounding tissues contribute a certain confinement blocking the creep of the gel and thus PIdx remains a useful piece of information for predicting the behaviour of the gel in vivo.

The measurement of the projection index is preferably carried out using a rheometer DHR2 equipped with a parallel-plate plate geometry (diameter 40 mm, anodised aluminium, TA Instruments®) combined with the TRIOS software (TA Instruments®). More particularly, the determination of the projection index can be carried out in the following manner. A bolus (sample) of 1 gram of hydrogel is deposited on a base plate of the rheometer. A pressure plate having a diameter of 40 mm applies a force on the sample, until its thickness, given by the distance between the base plate and the pressure plate, is equal to 700 μm (d₀).

Once the thickness has reached this predefined value do and, preferably, once the resistance force of the hydrogel is less than or equal to the value of the force F to be applied, a constant fixed compression force F of 2 N (Newton) is applied and the development of the variation of the thickness of the hydrogel is acquired. The acquisition starts by taking this instant as the time origin for the acquisition. This acquisition is carried out for 1 hour at 25° C. Preferably, the parameter d_{∞} is calculated using the OriginPro® software, version 9.6.0.172.

This software is used to apply a mathematical model in such a way as to have the best adjustment of the model with the development of thickness observed.

The model used is the generalised Maxwell model, which gives the development of the thickness of the hydrogel as a function of the time t by the formula:

$${d}_{gel} ( t ) = {d}_{∞} + \sum {A}_{i} \cdot {e}^{( - t / {τ}_{i} )}$$

Good results are obtained from the presence of two exponential terms:

$${d}_{gel} ( t ) = {d}_{∞} + {A}_{1} \cdot {e}^{( - t / {τ}_{1} )} + {A}_{2} \cdot {e}^{( - t / {τ}_{2} )}$$

d_{∞} corresponds to the thickness at equilibrium after infinite time, Aᵢ is a constant and τi a relaxation parameter, the number of members i of the equation being equal to 2.

The OriginPro® software enables the values of the Aᵢ and τi to be calculated/determined by iteration. Aᵢ and τi reflect a physical relaxation phenomenon of a hydrogel. These are positive values. In particular, Aᵢ (equivalent to a thickness) is greater than 0 and less than or equal to 700 μm. In particular, τi (equivalent to a time) is less than 24 hours, preferably less than 2 hours, more preferably less than 1 hour. In particular, the iteration is stopped when R²>0.99.

The projection index values are then calculated as follows:

$$PIdx ( \% ) = \frac{{d}_{∞}}{{d}_{0}} \cdot 100$$

The value d₀ corresponds to the initial thickness between the plates before application of the force. The fixed parameters for the application of the method for determining the projection index in order to characterise the hydrogel according to the invention, make it possible to easily discriminate the projection index values while applying a compression force in the LVER region of the so-called “volumiser” hydrogels.

### Hydrogels

The crosslinked polysaccharide-based hydrogels of the present invention have a modification ratio less than or equal to 1% and a projection index ranging from 50 to less than 80%, preferably ranging from 60 to less than 80%. The projection index is determined by the method detailed above.

Hydrogels having a projection index ranging from 50 to less than 80% have an ability to volumise tissues and sustainably maintain their thickness in the layers of the skin, including under the stress of facial movements. Such hydrogels are particularly useful as versatile filler gels, in other words useful for correcting moderate defects.

Furthermore, hydrogels having a modification ratio less than or equal to 1% are highly biocompatible.

The hydrogels according to the invention are crosslinked polysaccharide-based hydrogels. The hydrogels according to the present invention therefore comprise one or more crosslinked polysaccharide. The crosslinking makes it possible to obtain a hydrogel having the mechanical properties desirable for filling soft tissues.

The polysaccharide can be any polymer composed of monosaccharides joined together by glycosidic bonds, or the mixtures thereof. Preferably, the polysaccharide is chosen from pectin and pectic substances, chitosan; chitin; cellulose and its derivatives, agarose; glycosaminoglycans such as hyaluronic acid, heparosan, dermatan sulfate, keratan sulfate, chondroitin and chondroitin sulfate; and the mixtures thereof. Still more preferably, the polysaccharide is chosen from hyaluronic acid, heparosan, chondroitin and the mixtures thereof, yet more preferably the polysaccharide is hyaluronic acid or one of the salts thereof, in particular a physiologically acceptable salt such as the sodium salt, potassium salt, zinc salt, calcium salt, magnesium salt, silver salt, calcium salt, and the mixtures thereof. More particularly, the hyaluronic acid is in its acid form or in the form of a sodium salt (NaHA). The hydrogel can thus be a hydrogel based on hyaluronic acid and/or on one of its salts.

The polysaccharide generally has a weight average molar mass ranging from 0.03 to 10 MDa.

Preferably, if the polysaccharide is hyaluronic acid or one of the salts thereof, it has a weight average molar mass (Mw) ranging from 0.05 to 10 MDa, preferably ranging from 0.5 to 5 MDa, for example ranging from 0.07 to 10 MDa or from 0.07 to 5 MDa, or from 0.5 to 5 MDa or from 1 to 5 MDa or from 2 to 4 MDa.

### Crosslinking Agents

The polysaccharide can be crosslinked by means of a crosslinking agent, preferably chosen from bi- or multi-functional epoxy or non-epoxy crosslinking agents, in other words prepared by reaction of the polysaccharide with a crosslinking agent as described. The epoxy agents may include 1,4-butanediol diglycidyl ether (BDDE), 1,2,7,8-diepoxy-octane, 1,2-bis(2,3-epoxypropyl)-2,3-ethane (EGDGE), poly(ethyleneglycol)-diglycidyl ether (PEGDG), and the mixtures thereof. The non-epoxy agents may include endogenous polyamines, such as spermine, spermidine and putrescine, aldehydes such as glutaraldehyde, carbodiimides and divinyl sulfone, the derivatives of hydrazide such as adipic acid dihydrazide, bis-alkoxyamine, dithiol such as polyethylene glycol dithiol and the mixtures thereof. The non-epoxy agents may include amino acids such as cysteine, lysine; peptides or proteins containing amino acids such as cysteine, lysine; trimetaphosphates such as, for example, sodium trimetaphosphate, calcium trimetaphosphate, or even barium trimetaphosphate.

In certain embodiments, the crosslinking agent is an epoxy agent, preferably 1,4-butanediol diglycidyl ether (BDDE) or polyethyleneglycol-diglycidyl ether. The crosslinking agent is preferably 1,4-butanediol diglycidyl ether (BDDE).

In certain embodiments, the crosslinking agent is a non-epoxy agent, preferably chosen from the endogenous polyamines, aldehydes, carbodiimides, divinyl sulfone, amino acids, peptides and the mixtures thereof.

The hydrogel according to the present invention is preferably a crosslinked polysaccharide-based hydrogel, for which the molar crosslinking ratio is less than or equal to 1%, more preferably less than or equal to 0.8% or 0.5%, in particular ranging from 0.1% to 0.8% or from 0.1% to 0.5% (number of moles of crosslinking agent(s) per 100 moles of repetition unit of the one or more polysaccharides).

### Functionalisation Agent

The polysaccharide can be crosslinked by means of a functionalisation agent. The functionalisation agent enables a crosslinking of the polysaccharide by sol-gel reaction. Thus, the functionalisation agent typically comprises a single function capable of reacting with a functional group of the polysaccharide and comprises a silylated group capable of reacting with another silylated group via a sol-gel reaction so as to enable the crosslinking of the polysaccharide and to form a hydrogel.

The functionalisation agent is typically a molecule of formula Chem. II as shown below:

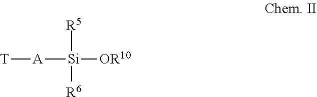

or a salt thereof,
wherein:
T represents an isocyanate, amino, epoxide, carboxyl, N-succinimidyloxycarbonyl, N-sulfosuccinimidyloxycarbonyl, halogenocarbonyl, isothiocyanate, vinyl, formyl, hydroxyl, sulfhydryl, hydrazino, acylhydrazino, aminoxy, carbodiimide group, or an acid anhydride residue; A represents a chemical bond or a spacer group;
R5 and R6, identical or different, represent a hydrogen atom; a halogen atom; an —OR4 group with R4 representing a hydrogen atom, an aryl group or an aliphatic hydrocarbon group having 1 to 6 carbon atoms; an aryl; or an aliphatic hydrocarbon group having 1 to 6 carbon atoms optionally substituted by one or more groups chosen from a halogen atom, an aryl and a hydroxyl;
R10 represents a hydrogen atom, an aryl group or an aliphatic hydrocarbon group having 1 to 6 carbon atoms.

Preferably, in formula Chem. II, T represents an isocyanate, sulfhydryl, amino, epoxide, vinyl, formyl, or carbodiimide group, more advantageously, T represents an epoxide or amino group, yet more advantageously T represents an epoxide group.

Preferably, in formula Chem. II, A represents a spacer group, more preferably a, in particular linear or branched and saturated, divalent aliphatic hydrocarbon chain, having 1 to 12 carbon atoms:
wherein one or more (in particular 1, 2, 3 or 4) divalent units are optionally intercalated between two carbon atoms of said chain, chosen from the arylenes, —O—, —S—, —S(O)—, —C(═O)—, —SO2- and —N(R9)- with R9 representing a hydrogen atom, an aliphatic hydrocarbon group having 1 to 6 carbon atoms, or an aryl-(C1-C6)alkyl group,
said chain being non-substituted or substituted by one or more monovalent groups chosen from a halogen atom, a hydroxyl, an aryl-(C1-C6)alkyl group.

Advantageously, A is an, in particular linear or branched and saturated, aliphatic divalent hydrocarbon chain, in which one or more —O— divalent units, more advantageously 1 to 4 —O— divalent units, yet more advantageously one divalent —O— unit, are optionally intercalated between two carbon atoms of said chain.

Preferably, A is a (C1-C12)alkylene chain, wherein one or more divalent —O— units, more preferably 1 to 4 divalent —O— units, yet more preferably one divalent —O— unit, are optionally intercalated between two carbon atoms of said chain.

In particular, A represents a divalent —(C1-C6)alkylene-O—(C1-C6)alkylene- chain, in particular —(C1-C4)alkylene-O—(C1-C4)alkylene-, more particularly a divalent —CH2-O—(CH2)3- chain, the CH2 group being bonded to T and the (CH2)3 group being bonded to Si in the molecule of formula Chem. II.

Advantageously, the spacer group will also make it possible to avoid steric hindrance between the silylated group and the T group of the molecule of formula Chem. II, ensuring a stable bond between these two groups.

Preferably, in formula Chem. II, R5 and R6, identical or different, represent an —OR4 group with R4 representing a hydrogen atom, an aryl group or an aliphatic hydrocarbon group having 1 to 6 carbon atoms; or an aliphatic hydrocarbon group having 1 to 6 carbon atoms optionally substituted by one or more groups chosen from a halogen atom, an aryl and a hydroxyl group.

In particular, R5 and R6, identical or different, represent an —OR4 group with R4 representing an (C1-C6)alkyl group; or a (C1-C6)alkyl group.

Advantageously, R5 and R6, identical or different, represent an —OR4 group with R4 representing a hydrogen atom, an aryl group or an aliphatic hydrocarbon group having 1 to 6 carbon atoms, preferably with R4 representing an aliphatic hydrocarbon group having 1 to 6 carbon atoms, such as a (C1-C6)alkyl group.

Preferably, in formula Chem. II, R10 represents a hydrogen atom or an aliphatic hydrocarbon group having 1 to 6 carbon atoms such as a (C1-C6)alkyl group, more advantageously R10 represents an aliphatic hydrocarbon group having 1 to 6 carbon atoms such as a (C1-C6)alkyl group.

Preferably, the molecule of formula Chem. II is such that:
T is as defined above and advantageously represents an amino or epoxide group, preferably an epoxide group;
A is a divalent —(C1-C6)alkylene-O—((C1-C6)alkylene- chain, in particular-(C1-C4)alkylene-O—(C1-C4)alkylene-, such as —CH2-O—(CH2)3-, the CH2 group preferably being bonded to T and the (CH2)3 group being bonded to Si in the molecule of formula Chem. II;
R5 and R6, identical or different, are each an —OR4 group with R4 representing a (C1-C6)alkyl group, preferably a methyl or an ethyl group; or a (C1-C6)alkyl group, preferably a methyl or a ethyl group; and
R10 is a (C1-C6)alkyl group, preferably a methyl or ethyl group;
the R5, R6 and OR10 groups being able to be identical.

In particular, the molecule of formula Chem. II is chosen from the (3-aminopropyl)triethoxysilane (APTES), (3-glycidyloxypropyl)trimethoxysilane (GPTMS), 3-3-glycidoxypropyldimethylethoxysilane, (3-glycidoxypropyldimethoxymethylsilane, glycidyloxypropyl)ethoxydimethoxysilane, (3-glycidyloxypropyl)triethoxysilane, diethoxy(3-glycidyloxypropyl)methylsilane, and the mixtures thereof; preferably from (3-glycidyloxypropyl)trimethoxysilane (GPTMS), (3-glycidyloxypropyl) ethoxydimethoxysilane, (3-glycidyloxypropyl)triethoxysilane, diethoxy(3-glycidyloxypropyl)methylsilane, and the mixtures thereof; yet more preferably (3-aminopropyl)triethoxysilane (APTES), (3-glycidyloxypropyl)trimethoxysilane (GPTMS) and the mixtures thereof.

The hydrogel according to the present invention is preferably a crosslinked polysaccharide-based hydrogel, the molar functionalisation ratio of which is less than or equal to 1%. More preferably, the hydrogel according to the present invention is a crosslinked polysaccharide-based hydrogel for which the molar functionalisation ratio ranges from 0.1 to 1% (number of moles of functionalisation agents per 100 moles of repetition unit of the one or more polysaccharides).

The hydrogel according to the present invention is preferably a crosslinked polysaccharide-based hydrogel, having a degree of modification (MOD) less than or equal to 1%.

The hydrogel according to the present invention advantageously has a degree of modification ratio (MOD)/PIdx less than or equal to 0.020, preferably less than or equal to 0.017, more preferably less than or equal to 0.015.

The hydrogel according to the present invention may comprise 0.1 to 5% by weight, preferably 1 to 3% by weight polysaccharide (for example hyaluronic acid), relative to the total weight of the hydrogel, the polysaccharide (for example hyaluronic acid) being present in crosslinked form. The hydrogel can further comprise a polysaccharide in non-crosslinked form.

In particular, the content of non-crosslinked polysaccharide (for example hyaluronic acid) can vary from 0.5 to 40% by weight, preferably 1 to 40% by weight, more preferably from 2 to 30% by weight, yet more preferably 2 to 20% relative to the total weight of polysaccharide (for example hyaluronic acid) present in the hydrogel.

The total concentration of polysaccharide in the hydrogel advantageously varies from 1 mg/g to 50 mg/g of hydrogel, more advantageously from 5 mg/g to 35 mg/g of hydrogel, yet more advantageously from 10 mg/g to 30 mg/g of hydrogel. This polysaccharide is preferably hyaluronic acid, yet more preferably sodium hyaluronate.

The hydrogel according to the present invention is preferably a sterile hydrogel, in particular sterilised by heat, typically at a plateau temperature of 121° C. to 135° C., preferably for a plateau duration ranging from 1 minute to 20 minutes with F0≥15. The sterilising value F0 corresponds to the time necessary, in minutes, at 121° C., to inactivate 90% of the population of microorganisms present in the product to be sterilised. Alternatively, the sterilisation can be carried out by radiation with gamma rays, UV or by means of ethylene oxide.

The hydrogel has a physiological pH, i.e. ranging from 6.8 to 7.8. The pH of the hydrogel according to the present invention is preferably greater than or equal to 6.9 and less than or equal to 7.4; 7.3; 7.2; 7.1 or 7.

The hydrogel according to the present invention is preferably an injectable hydrogel, in other words it can flow and be injected manually by means of a syringe equipped with a needle of diameter ranging from 0.1 to 0.5 mm, for example a 32 G, 30 G, 27 G, 26 G, 25 G hypodermic needle.

### Additional Components

In certain embodiments, the hydrogel of the present invention further comprises an additional component chosen from the group consisting of anaesthetics, antioxidants, amino acids, peptides, proteins such as elastin, vitamins, minerals, nucleic acids, nucleotides, nucleosides, co-enzymes, adrenergic derivatives, monohydrated and/or dihydrated sodium dihydrogen phosphate, sodium chloride and one of the mixtures thereof.

Non-crosslinked polysaccharides, in particular non-crosslinked hyaluronic acid, non-crosslinked heparosan or the mixture thereof, are examples of lubricating agents.

Examples of anaesthetics include, in a non-limiting manner, ambucaine, the amoxecaine, amyleine, aprindine, aptocaine, articaine, benzocaine, betoxycaine, bupivacaine, butacaine, butamben, butanilicaine, chlorobutanol, chloroprocaine, cinchocaine, clodacaine, cocaine, cryofluorane, cyclomethycaine, dexivacaine, diamocaine, diperodon, dyclonine, etidocaine, euprocine, febuverine, fomocaine, guafecainol, heptacaine, hexylcaine, hydroxyprocaine, hydroxytetracaine, isobutamben, leucinocaine, levobupivacaine, levoxadrol, lidamidine, lidocaine, lotucaine, menglytate, mepivacaine, meprylcaine, myrtecaine, octacaine, octodrine, oxetacaine, oxybuprocaine, parethoxycaine, paridocaine, phenacaine, piperocaine, piridocaine, polidocanol, pramocaine, prilocaine, procaine, propanocaine, propipocaine, propoxycaine, proxymetacaine, pyrrocaine, quatacaine, quinisocaine, risocaine, rodocaine, ropivacaine, tetracaine, tolycaine, trimecaine, and the salts thereof, in particular a hydrochloride salt, or a mixture of these.

Preferably, the hydrogel according to the invention comprises an anaesthetic as defined above and in particular lidocaine, mepivacaine or one of the salts thereof, such as the hydrochloride.

In certain embodiments, the hydrogel according to the invention comprises an anaesthetic, in particular mepivacaine, lidocaine or one of the salts thereof; more particularly a hydrochloride salt; preferably in quantities ranging from 0.1 to 30 mg/ml, for example 0.5 to 10 mg/ml or more preferably 2 to 5 mg/ml.

Examples of antioxidants include, in a non-limiting manner, glutathione, reduced glutathione, ellagic acid, spermine, resveratrol, retinol, L-carnitine, polyols, polyphenols, flavonols, theaflavins, catechins, caffeine, ubiquinol, ubiquinone, alpha-lipoic acid and their derivatives, and a mixture of these.

Examples of amino acids include, in a non-limiting manner, arginine (e.g., L-arginine), isoleucine (e.g., L-isoleucine), leucine (e.g., L-leucine), lysine (e.g., L-lysine or monohydrated L-lysine), glycine, valine (e.g., L-valine), threonine (e.g., L-threonine), proline (e.g., L-proline), methionine, histidine, phenylalanine, tryptophan, cysteine, their derivatives (e.g., N-acetyl derivatives such as N-acetyl-L-cysteine) and a mixture thereof.

Examples of vitamins and their salts include, in a non-limiting manner, vitamins E, A, C, B, especially vitamins B6, B8, B4, B5, B9, B7, B12, and preferably pyridoxine and its derivatives and/or salts, preferably pyridoxine hydrochloride.

Examples of minerals include, in a non-limiting manner, the salts of zinc (e.g., zinc acetate, in particular dehydrated zinc acetate, zinc citrate), magnesium salts, calcium salts (e.g., hydroxyapatite, in particular in bead form), the potassium salts, manganese salts, sodium salts, copper salts (e.g., copper sulphate, in particular pentahydrated copper sulphate), optionally in a hydrated form, and the mixtures thereof.

Examples of nucleic acids include, in a non-limiting manner, adenosine, cytidine, guanosine, thymidine, cytodine, their derivatives and a mixture thereof. Co-enzymes include the coenzyme Q10, CoA, NAD, NADP, and the mixtures thereof.

Adrenaline derivatives include adrenaline, noradrenaline and a mixture of these.

### Method for Preparing the Hydrogel

The hydrogel according to the invention is typically prepared by a method comprising the following steps:
a) providing at least one polysaccharide or a salt thereof;
b) providing at least one crosslinking agent or a salt thereof;
c) preparing a crosslinking reaction medium comprising the one or more polysaccharides, the one or more crosslinking agents and a solvent, the total quantity of crosslinking agent ranging from 0.001 to 0.01 mole per 1 mole of polysaccharide repetition unit, the duration of the preparation step of the reaction medium preferably not exceeding 5 hours; and
d) crosslinking reaction: placing the reaction medium obtained at the end of step c) under conditions enabling the crosslinking of the polysaccharide by reaction of the polysaccharide with the one or more crosslinking agents, namely at a pressure P less than or equal to atmospheric pressure and at a temperature T greater than the temperature of the eutectic point of the reaction medium as measured at pressure P and less than the temperature of the freezing point of the reaction medium as measured at pressure P, preferably for a duration ranging from 1 week to 17 weeks or from 2 to 17 weeks.

In a variant of the method for preparing the hydrogel described above, the crosslinking agent is replaced by a functionalisation agent. In such a variant, the crosslinking reaction d) is carried out by sol-gel reaction of the functionalised polysaccharide, the functionalised polysaccharide being obtained by reaction of the polysaccharide with the one or more functionalisation agents; the crosslinking reaction d) is carried out at a pressure P less than or equal to atmospheric pressure and at a temperature T greater than the temperature of the eutectic point of the reaction medium as measured at pressure P and less than the temperature of the freezing point of the reaction medium as measured at pressure P, preferably for a duration ranging from 1 week to 17 weeks or from 2 to 17 weeks. In such a variant of the method for preparing the hydrogel according to the invention, step b) comprises providing at least one functionalisation agent and step c) comprises preparing a reaction medium comprising the one or more polysaccharides, the one or more functionalisation agents and a solvent, the total quantity of functionalisation agent typically ranging from 0.001 to 0.01, preferably from 0.001 to 0.005 mole per 1 mole of polysaccharide repetition unit.

In such a variant of the method for preparing the hydrogel according to the invention, the total quantity of functionalisation agent in the reaction medium of step c) typically varies from 0.001 to 0.01 mole per 1 mole of polysaccharide repetition unit.

The crosslinked polysaccharide-based hydrogels prepared by the methods described above are highly biocompatible. Indeed, such hydrogels are prepared with low quantities of crosslinking agent. They nevertheless have mechanical properties suitable for the creation of volume.

### Providing at Least One Polysaccharide or a Salt Thereof (Step a))

Step a) of the method for preparing the hydrogel according to the invention comprises providing at least one polysaccharide or a salt thereof, in particular a physiologically acceptable salt thereof. The polysaccharide is as described above. Preferably, the polysaccharide is hyaluronic acid or a hyaluronic acid salt, preferably a sodium salt.

The polysaccharide can be provided in totally or partially hydrated form, or in dry form, such as in the form of a powder or fibres. More particularly, in step a), the polysaccharide is provided in dry form, such as in the form of a powder or fibres. When the polysaccharide is provided in hydrated form, it is in the form of a non-crosslinked gel or a solution. In particular, when the polysaccharide is in hydrated form, it is a non-crosslinked aqueous gel or an aqueous solution. More particularly, the polysaccharide is mixed with water, optionally with added phosphate buffer or supplemented phosphate buffer, in other words possibly comprising additional components as defined above. It should therefore be understood that the non-crosslinked aqueous gel or the aqueous solution of polysaccharide does not comprise sodium hydroxide.

Providing at Least One Crosslinking Agent or a Salt Thereof and/or Functionalisation Agent (Step b))

Step b) of the method for preparing the hydrogel according to the invention comprises providing at least one crosslinking agent or a salt thereof and/or at least one functionalisation agent, the functionalisation agent enabling crosslinking of the polysaccharide by sol-gel reaction.

The crosslinking agent and the functionalisation agent are as described above.

### Preparation of the Crosslinking Reaction Medium (Step c))

Step c) of the method for preparing the hydrogel according to the invention comprises preparing a crosslinking reaction medium. The reaction medium comprises one or more polysaccharides, one or more crosslinking agents and/or functionalisation agents and a solvent.

The solvent is typically water or a mixture comprising water and an organic solvent (typically a mixture comprising at least 90% by weight water, or at least 95% or at least 98% by weight water relative to the total weight of the solvent). For example, an organic solvent such as an alcohol, in particular ethanol, or DMSO, can be used to solubilise the crosslinking agent, for example when it involves poly(dimethylsiloxane) terminated at each end by a diglycidyl ether (CAS number: 130167-23-6), before its addition to the aqueous reaction medium.

The reaction medium can further comprise salts, pH adjusters, for example a Bronsted base, more preferably a hydroxide salt, such as sodium or potassium hydroxide, additional components as described above and the mixtures thereof. The addition of a Bronsted base may be particularly necessary when the functional groups of the crosslinking agent have an epoxide group or a vinyl group. In these cases, the crosslinking takes place at a pH greater than or equal to 10, more advantageously greater than or equal to 12, which requires the addition of a Bronsted base to the reaction medium (for example sodium hydroxide), typically at a concentration between 0.10 M and 0.30 M.

The total quantity of crosslinking agent in the reaction medium varies from 0.001 to 0.01 mole per 1 mole of polysaccharide repetition unit, yet more preferably from 0.001 to 0.008 or from 0.001 to 0.005 mole per 1 mole of polysaccharide repetition unit. When the polysaccharide is a glycosaminoglycan such as a hyaluronic acid, the repetition unit is a disaccharide unit.

The total quantity of functionalisation agent in the reaction medium typically varies from 0.001 to 0.01 mole or from 0.001 to 0.005 mole per 1 mole of polysaccharide repetition unit. For example, the total quantity of formula Chem. II or a salt thereof in the reaction medium typically varies from 0.001 to 0.01, preferably from 0.001 to 0.005 mole per 1 mole of polysaccharide repetition unit. Typically, the higher the weight average molar mass Mw of the polysaccharide, the lower will be the functionalisation ratio with a view to obtaining a hydrogel having equivalent mechanical properties, in particular equivalent viscoelastic properties (in particular elastic modulus G′, stress at the intersection of G′ and G″ and/or phase angle δ). In other words, the higher the weight average molar mass Mw of the polysaccharide, the lower will be the molar quantity of functionalisation agent in the reaction medium, for example in the molecule of formula Chem. II.

The functionalisation of the polysaccharide is typically carried out in an aqueous reaction medium.

The mass concentration of polysaccharide or polysaccharide salt in the reaction medium advantageously varies from 50 to 300 mg/g of solvent, preferably from 100 to 200 mg/g.

Step c) of the method typically comprises a homogenisation step of the reaction medium. The homogenisation is generally carried out by three-dimensional stirring, stirring with a mixer, stirring with blades or stirring with a spatula.

The reaction medium is typically prepared from polysaccharide or polysaccharides in dried form. When the reaction medium is prepared from polysaccharide or polysaccharides in hydrated form, the non-crosslinked aqueous gel or the aqueous polysaccharide solution used for the preparation of the reaction medium does not comprise sodium hydroxide.

Step c) is typically carried out at a temperature ranging from 4 to 35° C., preferably 15° C. to 25° C.

Preferably the duration of step c) does not exceed 5 hours. It generally varies from 15 minutes to 4 hours, preferably from 30 minutes to 2 hours. Thus, the maximum time of contact of the polysaccharide with sodium hydroxide before starting step d), whether the polysaccharide is provided in dried or hydrated form, is generally 5 hours, or from 15 minutes to 4 hours or from 30 minutes to 2 hours.

The reaction medium obtained at the end of step c) is advantageously placed directly under the conditions of step d) according to the invention.

### Crosslinking at Temperature T and Pressure P (Step d)

Crosslinking step d) of the above-described methods consists of crosslinking one or more polysaccharides. It consists of reacting the reaction medium in order to obtain a crosslinked polysaccharide-based hydrogel.

The crosslinking can be carried out by reaction of the one or more polysaccharides with the crosslinking agent (or by sol-gel reaction of the polysaccharide functionalised by reaction of the polysaccharide with the functionalisation agent).

The crosslinking is carried out by placing the reaction medium obtained at the end of step c) under conditions enabling the crosslinking of the polysaccharide, namely at a pressure P less than or equal to atmospheric pressure and at a temperature T greater than the temperature of the eutectic point of the reaction medium as measured at pressure P and less than the temperature of the freezing point of the reaction medium as measured at pressure P, preferably for a duration ranging from 1 week to 17 weeks or from 2 weeks to 17 weeks.

The pressure P is advantageously equal to atmospheric pressure or less than atmospheric pressure.

The temperature of the freezing point of the reaction medium designates the temperature at which the mixture of the components of the reaction medium, on the macroscopic scale, solidify, in other words become non-fluid. Below the freezing point, the mixture is in a frozen state which is characterised by the coexistence of components in solid and liquid form. The freezing state is maintained down to the temperature of the eutectic point of the reaction medium.

The temperature of the eutectic point of the reaction medium designates the temperature below which the mixture of the components of the reaction medium passes from a frozen state (coexistence of liquid and solid phases) to a completely solid state, in other words a state in which all the components of the mixture are in solid form. The freezing point and the eutectic point of a mixture depend on the pressure to which the mixture is subjected, therefore the freezing point and the eutectic point are measured at pressure P.

The freezing point and the eutectic point can be determined by differential scanning calorimetry. This method makes it possible to determine the phase transitions. For this purpose, the product to be studied is gradually cooled until its phase transitions are observed.

The temperature T is preferably greater than or equal to −55° C. and less than or equal to −5° C., preferably it ranges from −35° C. to −10° C. Still more preferably, the temperature T is approximately −20° C.

The pressure P is, preferably, atmospheric pressure. “Atmospheric pressure” is the pressure that is exerted by the air which constitutes the atmosphere on any surface in contact with it. It varies as a function of altitude. At an altitude of 0 m, the average air pressure is 101,325 Pa. Preferably, the pressure P is atmospheric pressure and the temperature T is greater than or equal to −55° C. and less than or equal to −5° C., preferably T varies from −35° C. to −10° C. or is approximately −20° C. The crosslinking of the polysaccharide according to step d) can be carried out under vacuum, in particular at a pressure P less than atmospheric pressure, preferably at a pressure P between 0.7.105 and 0.9.105 Pa (between 0.7 and 0.9 bar), preferably between 0.7.105 and 0.8.105 Pa (between 0.7 and 0.8 bar).

Step d) of crosslinking is typically performed for a duration of at least 1 hour, preferably at least 3 hours, preferably at least 72 hours, preferably at most 27 weeks.

Preferably, crosslinking step d) is performed for a duration ranging from 1 week to 25 weeks or from 1 to 20 weeks or from 1 to 17 weeks or else from 2 to 25 weeks, preferably ranging from 2 to 20 weeks or from 2 to 17 weeks, yet more preferably from 3 to 8 weeks or from 4 to 7 weeks and at temperature T and at pressure P.

The crosslinking of the polysaccharide mainly takes place during step d) (it can nevertheless start from step c)). This step therefore enables crosslinking of the polysaccharide chains with one another. The functional groups of the crosslinking agent react with functional groups present on the polysaccharides so as to bond the polysaccharide chains to one another and to crosslink them by forming intermolecular bonds. The crosslinking agent can also react with the functional groups present on a same polysaccharide molecule so as to form intramolecular bonds. Notably, the functional groups of the crosslinking agent react with —OH or —COOH, or even —CHO groups present on the polysaccharides, such as hyaluronic acid. Crosslinked polysaccharides comprising at least one crosslinking bond between two polysaccharide chains, said crosslinking bond being the residue of the crosslinking agent, are thus obtained.

The crosslinking can be carried out in the presence of a plurality of crosslinking agents. When the crosslinking is carried out in the presence of a plurality of crosslinking agents, the crosslinking agents can be added simultaneously or at separate times to the reaction medium. Step d) can thus comprise repeated crosslinking steps. The crosslinking is thus carried out in the presence of a total quantity of crosslinking agents ranging from 0.1 to 1 mole or 0.1 to 0.8 mole or 0.1 to 0.5 mole of crosslinking agents (or their salts) per 100 moles of polysaccharide repetition unit. The crosslinking conditions, in particular the percentage of crosslinking agent, duration and temperatures as well as the weight averaged molar masses (Mw) of the polysaccharide, used are interdependent.

The lower the percentage of crosslinking agent, the longer the duration of the reaction must be in order to obtain equivalent mechanical properties of the resultant gel. In other words, the lower the molar percentage of crosslinking agent, the fewer will be the reactive functions in the reaction medium and the less will be the probability that 2 groups meet and react together, thus the longer the duration of the reaction must be in order to enable the functions to react with one another and to form crosslinking bonds, and thus to obtain a hydrogel with desirable properties.

In a variant of the method for preparing the hydrogel, present step d) can be carried out at ambient temperature (20-25° C.) for a duration which can be shorter than that described above, for example for a duration ranging from 1 hour to 7 days.

### Sol-Gel Cross-Linking

### Functionalisation of the Polysaccharide

When the crosslinking is carried out by sol-gel reaction, the crosslinking reaction d) is carried out by sol-gel reaction of the functionalised polysaccharide, the functionalised polysaccharide being obtained by reaction of the polysaccharide with the one or more functionalisation agents. The functionalisation of the polysaccharide and the sol-gel reaction can be sequential or at least partially concomitant.

The polysaccharide is typically functionalised with at least one molecule of formula Chem. II as described above in such a way as to become a carrier of Si—OR groups which will be able to react together and lead to a crosslinked polysaccharide. Since the molecule of formula Chem. II comprises a single reactive function with regard to the polysaccharide and enables crosslinking only via a sol-gel reaction, it does not have the toxicity of conventional cross-linking agents: the molecule of formula Chem. II cannot directly crosslink with biological molecules (proteins, DNA, etc.). More specifically, the functional group T of the molecule of formula Chem. II reacts with a functional group present on the polysaccharides so as to functionalise the polysaccharide chains. Notably, the functional group T of the molecule Chem. II thus reacts with an —OH or —COOH group, or even a CHO function, present on the polysaccharides, such as hyaluronic acid. Functionalised polysaccharides are thus obtained, comprising dangling bonds on a polysaccharide chain, said dangling bonds comprising a -A-Si(R5)(R6)OR10 group, the -A-Si(R5)(R6)OR10 group originating from the molecule of formula Chem. II being able to give the hydrogel biological properties.

The solvent is typically water or a mixture comprising water and an organic solvent (for example an alcohol, in particular ethanol, or DMSO; typically a mixture comprising at least 90% by weight water, or at least 95% or at least 99% by weight water relative to the total weight of the solvent).

The reaction medium typically comprises 0.001 to 0.01, preferably 0.001 to 0.005 mole of the molecule of formula Chem. II or a salt thereof, per 1 mole of polysaccharide repetition unit.

The mass concentration of polysaccharide of the reaction medium is advantageously between 50 and 300 mg/g of solvent, preferably between 100 and 200 mg/g.

In certain embodiments, in particular when T is an epoxide, the functionalisation is carried out at a pH greater than or equal to 9, or greater than or equal to 10, more advantageously greater than or equal to 12, and in particular at a pH less than 14, for example less than or equal to 13.5. For this purpose, the reaction medium preferably comprises a Bronsted base, more preferably a hydroxide, yet more preferably sodium or potassium hydroxide. Advantageously, the reaction medium comprises sodium or potassium hydroxide at a concentration between 0.10 M and 0.30 M.

In certain embodiments, in particular when T is an amino group, the functionalisation is carried out at a pH less than 7, more advantageously greater than or equal to 4.5 and less than 7 or less than or equal to 6.5. For this purpose, the reaction medium preferably comprises a Bronsted acid, more preferably hydrochloric acid, sulfuric acid, or acetic acid.

In certain embodiments, the functionalisation of the polysaccharide is carried out at atmospheric pressure and at a temperature between 4° C. and 60° C., more preferably between 10° C. and 50° C.

In these embodiments, the duration of the functionalisation reaction can vary from 1 hour to 2 weeks, more particularly from 3 hours to 1 week, yet more particularly from 3 hours to 96 hours, for example from 3 hours to 80 hours, in particular from 3 hours to 75 hours.

In certain embodiments, in particular when the functionalisation and crosslinking of the polysaccharide are concomitant or partially concomitant, the functionalisation of the polysaccharide can be at least partially carried out at a temperature T greater than the temperature of the eutectic point of the reaction medium as measured at pressure P and less than the temperature of the freezing point of the reaction medium as measured at pressure P. Here, the pressure P is advantageously atmospheric pressure.

The higher the functionalisation temperature, the shorter will be the functionalisation time in order to obtain the same degree of modification by functionalisation.

### Sol-Gel Reaction

The “sol-gel reaction” consists of forming Si—O—Si bonds from Si—OR groups, with R representing a hydrogen atom, an aryl group or an aliphatic hydrocarbon group having 1 to 6 carbon atoms. This reaction proceeds as follows:
(i) if R is not a hydrogen atom, a hydrolysis step of at least some of the Si—OR groups in order to give Si—OH groups; then
(ii) a step of condensing Si—OH groups two-by-two or a Si—OH group with a Si—OR group in order to form Si—O—Si bonds.

The functionalised polysaccharide is crosslinked by sol-gel reaction in order to give a hydrogel.

This step enables the polysaccharide chains to be cross-linked with one another when they are functionalised with molecules of formula Chem. II. More specifically, during this step, at least some of the Si—OR10 groups and optionally at least some of the SiOR4 groups will react two-by-two, optionally after hydrolysis of these groups, to form Si—O—Si bonds. This implies that two molecules of formula Chem. II grafted on the polysaccharide chains will react together via their Si—OR 10 (or SiOR4, as applicable) terminal groups and bond covalently via the formation of a Si—O—Si bond thus enabling bonding together of the polysaccharide chains and their crosslinking.

In this way, crosslinked polysaccharides are obtained, comprising crosslinking bonds between two polysaccharide chains, said crosslinking bonds comprising a divalent —Si—O—Si— group.

The pressure P is advantageously less than or equal to atmospheric pressure.

The crosslinking of the polysaccharide according to step d) can be carried out under vacuum, in particular at a pressure P less than atmospheric pressure, preferably at a pressure P between 0.7.105 and 0.9.105 Pa (between 0.7 and 0.9 bar), preferably between 0.7.105 and 0.8.105 Pa (between 0.7 and 0.8 bar).

The pressure P is advantageously atmospheric pressure.

In certain embodiments, the polysaccharide can be crosslinked by a crosslinking agent and by sol-gel reaction (by means of a functionalisation agent). The reactions can be successive or concomitant.

### Additional Steps

The method for preparing the hydrogel according to the invention can comprise one or more additional steps, such as steps of adding one or more additional components, purification, sterilisation, sieving, swelling and/or packaging.

The method for preparing the hydrogel according to the invention can comprise a step of adding at least one additional component. The additional component can be chosen from lubricants; cosmetic active ingredients such as antioxidants, co-enzymes, amino acids, vitamins, minerals and nucleic acids; therapeutic active ingredients such as anaesthetics, antibiotics, antifungals and adrenaline and its derivatives, and the mixtures thereof. The additional components can be as described above.

The method for preparing the hydrogel according to the invention can comprise at least one purification step. The purification can be carried out by dialysis.

The method for preparing the hydrogel according to the invention can comprise a step of sterilising the hydrogel. The sterilisation is preferably carried out by heat. The sterilisation is generally carried out by increasing the temperature of the sterilisation medium up to a so-called “plateau temperature”, which is maintained for a determined so-called “plateau duration”. The sterilisation is preferably carried out at a plateau temperature ranging from 121° C. to 135° C., preferably for a plateau duration ranging from 1 minute to 20 minutes with F0≥15. The sterilising value F0 corresponds to the time necessary, in minutes, at 121° C., to inactivate 90% of the population of microorganisms present in the product to be sterilised. Alternatively, the sterilisation can be carried out, in particular, by radiation with gamma rays, UV or by means of ethylene oxide.

The method can comprise a step of sieving the hydrogel, more particularly with a sieve having a porosity between 50 and 2000 μm. This sieving step makes it possible to obtain a more homogeneous hydrogel with the most constant possible extrusion force, i.e., the most regular possible extrusion force. A person skilled in the art knows to select a sieve with suitable pore size depending on the mechanical properties of the hydrogel undergoing preparation.

The method can comprise a step of swelling the hydrogel. During the step of swelling the hydrogel, the concentration of polysaccharide of the hydrogel is adjusted.

In particular, a solvent is added, for example water, a phosphate buffer, water for an injectable preparation. More particularly, the added solvent has a pH that is approximately the physiological pH (6.8-7.8). The concentration of polysaccharide obtained in the hydrogel following the swelling step advantageously varies from 1 mg/g to 50 mg/g of hydrogel, more advantageously from 5 mg/g to 35 mg/g of hydrogel, yet more advantageously from 10 mg/g to 30 mg/g of hydrogel.

The method can comprise a step of packaging the hydrogel, for example in an injection device.

Where applicable, the step of adding one or more additional components preferably takes place after the purification step.

Where applicable, the step of addition of one or more additional components preferably takes place before the sterilisation step.

In particular, the step of adding one or more additional components can also comprise adding at least one therapeutic activity ingredient, or at least one cosmetic active ingredient, or the mixture thereof. When at least one therapeutic active ingredient and/or at least one cosmetic active ingredient is added, the step of adding one or more additional components preferably takes place after step d).

Where applicable, the purification step preferably takes place after the step of adding one or more additional components.

Where applicable, the purification step preferably takes place before the sterilisation step.

Where applicable, the purification step preferably takes place before the sieving step.

The sterilisation step is preferably carried out after steps a) to d) and the optional additional steps. In particular, the hydrogel is sterilised after having been packaged in its injection device and the packaging of the hydrogel takes place following all the steps of the method and before sterilisation.

### Applications

The hydrogel according to the invention can be injected using any one of the methods known to a person skilled in the art. In particular, a hydrogel according to the invention can be administered by means of an injection device suitable for an intraepidermal and/or intradermal and/or subcutaneous and/or supraperiosteal injection. The injection device can be chosen, in particular, from a syringe, a set of microsyringes, a wire, a laser or hydraulic device, an injection gun, a needle-free injection device, or a microneedle roller.

The present invention also relates to an injection device comprising a hydrogel according to the invention.

The hydrogel according to the invention is preferably injected subcutaneously.

The hydrogel according to the invention can have therapeutic and/or cosmetic applications.

In certain embodiments, the present invention also relates to a hydrogel for non-therapeutic use in the filling and/or replacement of tissues, in particular soft tissues, in particular by injection of the hydrogel into the tissue.

In the cosmetic field, the hydrogel according to the invention can be used, in particular, to compensate for volume loss of the tissues due to ageing.

The hydrogel according to the present invention can be used in the prevention and/or cosmetic treatment of a change in the surface appearance of the skin. For example, the hydrogel according to the invention can be used in the cosmetic field for preventing and/or treating the change in the viscoelastic or biomechanical properties of the skin; for filling volume defects of the skin, in particular for filling wrinkles, fine lines and scars; for attenuating the nasolabial folds and bitterness folds; for increasing the volume of the cheekbones, the chin or lips; for restoring the volumes of the face, in particular the cheeks, temples, the oval of the face, and around the eye; for reducing the appearance of wrinkles and fine lines.

The present invention also relates to the cosmetic use of a hydrogel as previously described for filling tissues, in particular soft tissues, in particular to compensate for volume losses of the tissues due to ageing.

### EXAMPLES

### 1. Equipment and Materials

1.5 MDa, non-cross-linked sodium hyaluronate,
BDDE,
0.25 M NaOH,
Divinyl sulfone (DVS),
1 M HCl,
Phosphate buffer,
Lidocaine hydrochloride,
Three-dimensional stirrer,
Rheometer DHR-2-TA Instrument®,
Dynamometer and test bench,
Paddle mill moderniser,
Polyethylene sterile bags.

### 2. Methods

Unless otherwise stated, the steps described below are carried out at ambient temperature (21° C.).

### Example 1

### Preparation of a Hydrogel According to the Invention (Hydrogel A)

DVS and sodium hyaluronate (1.5 MDa, 120 mg/g) are dissolved in a 0.25 M aqueous solution of sodium hydroxide, in a sterile bag. The mixture was then homogenised in a blade mill until the sodium hyaluronate was completely dissolved (i.e. until the absence of agglomerate and obtaining of a uniform colour). The mixture obtained is maintained at atmospheric pressure and at the temperatures and for the duration presented in Table 1. The pH of the mixture obtained was approximately 13.

A 1 N solution of HCl was then added to the sterile bag until a pH of 7.3±0.5 was obtained. The mixture obtained is diluted with the phosphate buffer solution PBS to a concentration of 23 mg of hyaluronic acid per gram of product for Gel B. The mixture obtained is homogenised for 24 hours by means of a three-dimensional stirrer. The mixture is dialysed.

A solution of sodium hyaluronate in the non-crosslinked form with high molecular weight was then added as a lubricant.

An aqueous solution of lidocaine hydrochloride is added to obtain 0.3% by weight lidocaine hydrochloride relative to the weight of the resulting product.

The product thus obtained was sieved on the order of a micron then packaged in a syringe.

Finally, the product was sterilised with an autoclave (plateau temperature between 121° C. and 135° C. with F0≥15).

The crosslinking conditions of the hydrogels according to the invention are presented in table 1 below.

TABLE 1
crosslinking conditions

| | Molar TR | Crosslinking | Crosslinking |
|---|---|---|---|
| Reference | DVS (%) | time (in hours) | temperature (in ° C.) |
| Hydrogel A | 0.5 | 4 | 21 |

The generalised Maxwell model is applied, which gives the development of the thickness of the hydrogel as a function of time t by the formula:

$${d}_{gel} ( t ) = {d}_{∞} + \sum {A}_{i} \cdot {e}^{( - t / {τ}_{i} )}$$

where d_{∞} is the limit thickness obtained at equilibrium, Aᵢ is a constant and τi a relaxation parameter, the number of members I of the equation being equal to 2.

The results are obtained with the presence of two exponential terms:

$${d}_{gel} ( t ) = {d}_{∞} + {A}_{1} \cdot {e}^{( - t / {τ}_{1} )} + {A}_{2} \cdot {e}^{( - t / {τ}_{2} )}$$

The projection index values are then calculated as follows:

$$PIdx ( \% ) = \frac{{d}_{∞}}{{d}_{0}} \cdot 100$$

d₀ being the initial thickness is 700 μm and do being obtained through the application of the generalised Maxwell model.

The MOD of the hydrogel is measured by 1H NMR.

The results are presented in table 2 below.

TABLE 2
results

| | | Projection | |
|---|---|---|---|
| Reference | MOD (%) | index Pidx (%) | MOD/Pidx |
| Hydrogel A | <1% | 57% | 0.017 |

The hydrogel according to the invention, hydrogel A, has a projection index PIdx in the range from 50% to less than 80%, reflecting a good capacity for creating volume and filling tissues. The hydrogel according to the invention has a low MOD (<1%) reflecting a strong biocompatibility.

### Example 2

A hydrogel according to the invention (Hydrogel B) is compared with commercial 28ydrazine hydrogels Juvederm Voluma®—20 mg/g (Allergan®), Teosyal RHA®4-23 mg/g (Teoxane®), Belotero® Volume—26 mg/g (Merz Pharma®), Restylane® Lyft—20 mg/g (Galderma®). The five hydrogels compared are sterile.

### Preparation of a Hydrogel According to the Invention (Hydrogel B)

BDDE and sodium hyaluronate (1.5 Mda, 120 mg/g) were dissolved in a 0.25 M aqueous solution of sodium hydroxide, in a sterile bag. The mixture was then homogenised in a blade mill until the sodium hyaluronate was completely dissolved (i.e. until a transparent and perfectly homogeneous product (i.e., absence of any agglomerate) was obtained). The mixture obtained is maintained at atmospheric pressure and at the temperatures and for the duration presented in Table 3. The pH of the mixture obtained was approximately 13.

A 1 N solution of HCl was then added to the sterile bag until a pH of 7.3±0.5 was obtained. The mixture obtained is diluted with the phosphate buffer solution PBS to a concentration of 18 mg of hyaluronic acid per gram of product for hydrogel B. The mixture obtained is homogenised for 24 hours by means of a three-dimensional stirrer. The mixture is dialysed.

A solution of sodium hyaluronate in the non-crosslinked form with high molecular weight was then added as a lubricant.

An aqueous solution of lidocaine hydrochloride is added to obtain 0.3% by weight lidocaine hydrochloride relative to the weight of the resulting product.

The product thus obtained was sieved on the order of a micron then packaged in a syringe.

Finally, the product was sterilised with an autoclave (plateau temperature between 121° C. and 135° C. with F0≥15).

The crosslinking conditions of the hydrogels according to the invention are presented in table 3 below.

TABLE 3
crosslinking conditions

| | Molar TR | Crosslinking | Crosslinking |
|---|---|---|---|
| Reference | BDDE (%) | time (in days) | temperature (in ° C.) |
| Hydrogel B | 0.5 | 21 | −20° C. |

The force of 2 newtons applied in the method for determining the projection index is included in the LVER interval of the 5 hydrogels to be evaluated. The projection index values for these 5 hydrogels are measured at 25° C. using a rheometer DHR2 equipped with a parallel-plate plate geometry (diameter 40 mm, anodised aluminium, TA Instruments®) combined with the TRIOS software (TA Instruments®). 1 gram of hydrogel is applied between the plates and the gap between these plates corresponds to an initial thickness of 700 μm. A compression force of 2 newtons is then applied for 1 hour and the development in the variation of the thickness of the hydrogel is acquired. The parameter d_{∞} is calculated using the OriginPro® 2019 software, version 9.6.0.172.

The generalised Maxwell model is applied, which gives the development of the thickness of the hydrogel as a function of time t by the formula:

$${d}_{gel} ( t ) = {d}_{∞} + \sum {A}_{i} \cdot {e}^{( - t / {τ}_{i} )}$$

where d_{∞} is the limit thickness obtained at equilibrium, Aᵢ is a constant and τi a relaxation parameter, the number of members I of the equation being equal to 2.

The results are obtained with the presence of two exponential terms:

$${d}_{gel} ( t ) = {d}_{∞} + {A}_{1} \cdot {e}^{( - t / {τ}_{1} )} + {A}_{2} \cdot {e}^{( - t / {τ}_{2} )}$$

The projection index values are then calculated as follows:

$$PIdx ( \% ) = \frac{{d}_{∞}}{{d}_{0}} \cdot 100$$

d₀ being the initial thickness is 700 μm and do being obtained through the application of the generalised Maxwell model.

The MOD of each of the hydrogels are measured by 1H NMR. The hydrogel is precipitated in isopropanol and dried for 6 hours under vacuum. The resulting residues of hyaluronic acid are dissolved to 10 mg/mL in D2O. 50 μL of hyaluronidase (Type VI-S from bovine testes, 3 kU/mL in D2O) were added in order to degrade the gels, for 18 hours at 37° C. The analysis is carried out on a 400 MHz Bruker Avance spectrometer. The MOD is determined according to the following equation:

$$MOD ( \% ) = ( {I}^{δ H 1.6 - 1.7} / 4 ) / ( {I}^{δ H 2. - 2.1} / 3 ) \cdot 100.$$

The minimum quantification limit of the MOD is 1%.

The results are presented in table 4 below.

TABLE 4
results.

| | | Projection | |
|---|---|---|---|
| Reference | MOD (%) | index Pidx (%) | MOD/Pidx |
| Hydrogel B | <1% | 67% | <0.014 |
| Teosyal RHA ® 4 | 4% | 78% | 0.051 |
| (Teoxane ®) | | | |
| Belotero ® Volume | 8% | 47% | 0.170 |
| (Merz Pharma ®) | | | |
| Restylane Lyft | >1% | 38% | 0.026 |
| (Galderma ®) | | | |
| Juverdem Voluma ® | 6% | 65% | 0.092 |
| (Allergan ®) | | | |

The hydrogel according to the invention, hydrogel B, has a projection index PIdx in the range from 50% to less than 80%, reflecting a good capacity for creating volume and filling tissues. Unlike the commercial hydrogels, the hydrogel according to the invention as a very low MOD (<1%) reflecting a strong biocompatibility.

1. A crosslinked polysaccharide-based hydrogel having a modification ratio less than or equal to 1% and having a projection index PIdx ranging from 50% to less than 80%, the projection index being determined according to a method comprising the following steps:
1) subjecting a bolus of one gram of hydrogel to a fixed constant compression force F of 2 newtons, said hydrogel being disposed between two circular pressure plates of a rheometer and having an initial thickness d0 of 700 μm given by the distance between the two plates, the two plates being parallel and having a diameter of 40 mm;
2) acquiring, at 25° C., the development of the variation of the thickness of the hydrogel compressed in this way for 1 hour;
3) parameterising a generalised Maxwell model approximating the development observed from the acquisition carried out in step 2), said model expressing the thickness of the hydrogel as a function of time d_{gel}(t) and responding to the following equation 1:
$${d}_{gel} ( t ) = {d}_{∞} + \sum {A}_{i} \cdot {e}^{( - t / {τ}_{i} )} \qquad ( equation 1 )$$
where d_{∞} is the limit thickness obtained at equilibrium, Aᵢ is a constant and τi a relaxation parameter, the number of members I of the equation being equal to 2,
4) determining, from the model, a limit thickness do towards which the hydrogel tends to develop over time,
5) generating the projection index PIdx from the following equation 2:
$$PIdx ( \% ) = \frac{{d}_{∞}}{{d}_{0}} \cdot 100. \qquad ( equation 2 )$$

2. The hydrogel according to claim 1, wherein the hydrogel has a degree of modification ratio (MOD)/PIdx less than or equal to 0.020.

3. The hydrogel according to claim 1, having a projection index PIdx ranging from 60% to less than 80%.

4. The hydrogel according to claim 1 further comprising a non-crosslinked polysaccharide.

5. The hydrogel according to claim 1 wherein the crosslinked polysaccharide represents 0.1 to 5% by weight relative to the total weight of the hydrogel.

6. The hydrogel according to claim 1 wherein the crosslinked polysaccharide is prepared by reaction of the polysaccharide with a crosslinking agent selected from the group consisting of 1,4-butanediol diglycidyl ether (BDDE), 1,2,7,8-diepoxy-octane, poly(ethylene glycol) diglycidyl ether (PEGDG), 1,2-bis(2,3-epoxypropoxy) ethane (EGDGE), and the mixtures thereof.

7. The hydrogel according to claim 1 wherein the crosslinked polysaccharide is prepared by sol-gel reaction of a polysaccharide functionalised by means of a functionalisation agent of formula Chem. II 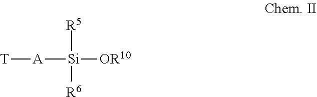
or a salt thereof,
wherein:
T represents an isocyanate, amino, epoxide, carboxyl, N-succinimidyloxycarbonyl, N-sulfosuccinimidyloxycarbonyl, halogenocarbonyl, isothiocyanate, vinyl, formyl, hydroxyl, sulfhydryl, hydrazino, acylhydrazino, aminoxy, carbodiimide group, or an acid anhydride residue;
A represents a chemical bond or a spacer group;
R⁵ and R⁶, identical or different, represent a hydrogen atom; a halogen atom; an —OR⁴ group with R⁴ representing a hydrogen atom, an aryl group or an aliphatic hydrocarbon group having 1 to 6 carbon atoms; an aryl; or an aliphatic hydrocarbon group having 1 to 6 carbon atoms optionally substituted by one or more groups chosen from a halogen atom, an aryl and a hydroxyl; and
R¹⁰ represents a hydrogen atom, an aryl group or an aliphatic hydrocarbon group having 1 to 6 carbon atoms.

8. A method for the preparation of a hydrogel according to claim 1 comprising the following steps:
a) providing at least one polysaccharide or a salt thereof;
b) providing at least one crosslinking agent or a salt thereof;
c) preparing a crosslinking reaction medium comprising the one or more polysaccharides, the one or more crosslinking agents and a solvent, the total quantity of crosslinking agent ranging from 0.001 to 0.01 mole per 1 mole of polysaccharide repetition unit, the duration of the preparation step of the reaction medium preferably not exceeding 5 hours; and
d) placing the reaction medium obtained at the end of step c), at a pressure P less than or equal to atmospheric pressure and at a temperature T greater than the temperature of the eutectic point of the reaction medium as measured at pressure P and less than the temperature of the freezing point of the reaction medium as measured at pressure P, preferably for a duration ranging from 1 week to 17 weeks.

9. A method for the preparation of a hydrogel according to claim 1 comprising the following steps:
a) providing at least one polysaccharide or a salt thereof;
b) providing at least one functionalisation agent or a salt thereof, of formula Chem. II 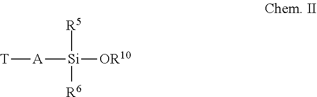
or a salt thereof,
wherein:
T represents an isocyanate, amino, epoxide, carboxyl, N-succinimidyloxycarbonyl, N-sulfosuccinimidyloxycarbonyl, halogenocarbonyl, isothiocyanate, vinyl, formyl, hydroxyl, sulfhydryl, hydrazino, acylhydrazino, aminoxy, carbodiimide group, or an acid anhydride residue;
A represents a chemical bond or a spacer group;
R⁵ and R⁶, identical or different, represent a hydrogen atom; a halogen atom; an —OR⁴ group with R⁴ representing a hydrogen atom, an aryl group or an aliphatic hydrocarbon group having 1 to 6 carbon atoms; an aryl; or an aliphatic hydrocarbon group having 1 to 6 carbon atoms optionally substituted by one or more groups chosen from a halogen atom, an aryl and a hydroxyl; and
R¹⁰ represents a hydrogen atom, an aryl group or an aliphatic hydrocarbon group having 1 to 6 carbon atoms;
c) preparing a crosslinking reaction medium comprising the one or more polysaccharides, the one or more functionalisation agents and a solvent, the total quantity of functionalisation agent ranging from 0.001 to 0.01 mole per 1 mole of polysaccharide repetition unit, the duration of the preparation step of the reaction medium preferably not exceeding 5 hours; and
d) placing the reaction medium obtained at the end of step c), at a pressure P less than or equal to atmospheric pressure and at a temperature T greater than the temperature of the eutectic point of the reaction medium as measured at pressure P and less than the temperature of the freezing point of the reaction medium as measured at pressure P, preferably for a duration ranging from 1 week to 17 weeks.

10. The hydrogel according to claim 1, wherein the crosslinked polysaccharide is hyaluronic acid, a hyaluronic acid salt or the mixtures thereof.

11. The hydrogel according to claim 1 comprising a total polysaccharide concentration in the hydrogel varying from 1 mg/g to 50 mg/g of hydrogel.

12. The hydrogel according to claim 1 in a sterile form.

13. The hydrogel according to claim 1 in an injectable form.

14. The hydrogel according to claim 1 further comprising an anaesthetic agent.

15. A cosmetic method for preventing and/or treating the alterations in viscoelastic or biomechanical properties of the skin; to fill wrinkles, fine lines and scars; to reduce nasolabial folds and bitterness folds; to increase the volume of cheekbones, of the chin or lips, or to reduce the appearance of wrinkles and fine lines comprising administering to a subject a hydrogel according to claim 1.

16. The hydrogel of claim 1 wherein the hydrogel has a degree of modification ratio (MOD)/PIdx less than or equal to 0.017.

17. The hydrogel of claim 4 wherein the non-crosslinked polysaccharide is selected from the group consisting of non-crosslinked hyaluronic acid, non-crosslinked heparosan or the mixture thereof.

18. The hydrogel of claim 1 wherein the crosslinked polysaccharide represents 1 to 3% by weight, relative to the total weight of the hydrogel.

19. The hydrogel of claim 1 comprising a total polysaccharide concentration in the hydrogel varying from 5 mg/g to 35 mg/g of hydrogel.

20. The hydrogel of claim 1 comprising a total polysaccharide concentration in the hydrogel varying from 10 mg/g to 30 mg/g of hydrogel.
